(19) United States
(12) Patent Application Publication
(10) Pub. No.: US 2026/0108466 A1
(43) Pub. Date: Apr. 23, 2026

(54) VARENICLINE FORMULATIONS

(71) Applicant: Pfizer Inc., New York, NY (US)

(72) Inventors: Alan Francis CARMODY, Sandwich, Kent (GB); Alastair John COUPE, Sandwich, Kent (GB)

(21) Appl. No.: 19/476,433

(22) PCT Filed: Apr. 22, 2024
(86) PCT No.: PCT/IB2024/053911

§ 371 Date: Oct. 17, 2025

Related U.S. Application Data
(60) Provisional application No. 63/497,877, filed on Apr. 24, 2023.

(51) Int. Cl. A61K 9/20; A61K 9/28; A61K 31/55

(52) U.S. Cl. CPC A61K 9/2013; A61K 9/2009; A61K 9/2054; A61K 9/282; A61K 9/2893; A61K 31/55

(57) ABSTRACT

In an embodiment, there is provided a varenicline formulation consisting essentially of about 0.85% varenicline tartrate and greater than 1.75% to about 3.0% propyl gallate, wherein the formulation maintains a level of N-nitrosovarenicline (NNV) impurity over the shelf-life of the formulation of not more than 18.5 ppm (37 ng/day) of acceptable daily intake (ADI). In another embodiment, there is provided a varenicline formulation comprising a tablet core comprising about 0.85% varenicline tartrate, about 60.50% microcrystalline cellulose, about 33.33% dibasic calcium phosphate anhydrous, about 2.00% croscarmellose sodium, about 2.57% propyl gallate, and about 0.75% magnesium stearate; and a film coating.

### BACKGROUND OF THE INVENTION

The present invention relates to varenicline formulations and methods for their manufacture. The varenicline formulations are designed to minimizes the risk of nitrosamine formation within the active pharmaceutical ingredient and the drug product.

Pharmaceutical compounds with secondary dialkylamino and trialkylamino groups are known to have risks associated with the formation of toxic N-nitroso derivatives (i.e., nitrosamines) upon reaction with various nitrosating agents (e.g., nitrites and nitrates) that may be present in the active pharmaceutical ingredient or in the manufacture of the final drug product. In humans, nitrosamines tend to be the most common N-nitroso compounds observed and have been linked to cancers such as kidney, liver and lung cancer. New drugs and pharmaceutical excipients are accordingly screened in bacterial mutagenicity assays at a preclinical stage of drug development in order to identify any potential toxicity of this kind.

The U.S. FDA has identified seven nitrosamine impurities that could be present in drug products: N-nitrosodimethylamine (NDMA), N-nitrosodiethylamine (NDEA), N-nitroso-N-methyl-4-aminobutanoic acid (NMBA), N-nitrosoisopropylethyl amine (NIPEA), N-nitrosodiisopropylamine (NDIPA), N-nitrosodibutylamine (NDBA), and N-nitrosomethylphenylamine (NMPA). Five of them (NDMA, NDEA, NMBA, NIPEA, and NMPA) have actually been detected in drug substances or drug products. Some of these impurities have been classified as potent genotoxic agents in several animal species and some are classified as probable or possible human carcinogens by the International Agency for Research on Cancer (IARC). As a result, the FDA has published interim acceptable limits for these impurities.

The need to minimize the exposure of humans to N-nitroso compounds, and therefore to nitrite, has been increasingly recognized in recent years and the level of these compounds in active pharmaceutical ingredients and marketed drug products has been subject to increasing levels of scrutiny. Many common excipients used in the formulation of drugs contain ppm levels of nitrite and other nitrosating agents and consequently have the potential to lead to the formation nitrosamines in the drug product in excess of the required acceptable daily intake level.

Therefore, a need exists to provide drug products that are nitrosamine free or drug products that contain acceptable daily intake levels of nitrite and other nitrosating agents. The present invention provides a solution to this problem by utilizing antioxidants in the drug product formulations described herein which provides the advantage of a drug product that has regulatory-defined acceptable levels of nitrosamines, and maintains these levels over time, or remain nitrosamine free over time. One such product is varenicline. It has been found that the addition of an antioxidant (e.g., propyl gallate) and removal of compendial excipients (e.g., colloidal silicon dioxide) from the original varenicline commercial formulation provides a varenicline drug product that contains acceptable levels of nitrosamine impurities and minimizes the increase in nitrosamine levels over time.

### SUMMARY OF THE INVENTION

The present invention is directed to varenicline formulations wherein the formulations maintain a level of N-nitrosovarenicline (NNV) impurity over the shelf-life of the formulation of not more than 18.5 ppm (37 ng/day) of acceptable daily intake (ADI). The present invention also provides, in part, methods of for reducing nicotine addiction or aiding in the cessation or lessening of tobacco use in a mammal by administration of the varenicline formulations described herein.

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Described below are embodiments of the invention. It is to be understood that both the foregoing general description and the following detailed description are exemplary and explanatory only and are not restrictive of the invention, as claimed.

### BRIEF DESCRIPTION OF THE DRAWINGS

FIG. 1 shows a comparison of drug-release profiles of varenicline tartrate 0.5 mg tablets PG (propyl gallate) and Chantix® IR tablets, 0.5 mg in 0.1N HCl.

FIG. 2 shows a comparison of drug-release profiles of varenicline tartrate 1.0 mg tablets PG (propyl gallate) and Chantix® IR tablets, 1.0 mg in 0.1N HCl.

FIG. 3 shows a comparison of drug-release profiles of varenicline tartrate 0.5 mg tablets PG (propyl gallate) and Chantix® IR tablets, 0.5 mg in pH 4.5 acetate.

FIG. 4 shows a comparison of drug-release profiles of varenicline tartrate 1.0 mg tablets PG (propyl gallate) and Chantix® IR tablets, 1.0 mg in pH 4.5 acetate.

FIG. 5 shows a comparison of drug-release profiles of varenicline tartrate 0.5 mg tablets PG (propyl gallate) and Chantix® IR tablets, 0.5 mg in pH 6.8 phosphate.

FIG. 6 shows a comparison of drug-release profiles of varenicline tartrate 1.0 mg tablets PG (propyl gallate) and Chantix® IR tablets, 1.0 mg in pH 6.8 phosphate.

### DETAILED DESCRIPTION OF THE INVENTION

The present invention may be understood more readily by reference to the following detailed description of exemplary embodiments of the invention and the examples included therein. It is to be understood that this invention is not limited to the specific examples or the methods of making that may of course vary.

It is to be understood that the terminology used herein is for the purpose of describing particular embodiments only and is not intended to be limiting.
E1 According to a first embodiment of the invention there is provided a varenicline formulation consisting essentially of about 0.85% varenicline tartrate and greater than 1.75% to about 3.0% propyl gallate, wherein the formulation maintains a level of N-nitrosovarenicline (NNV) impurity over the shelf-life of the formulation of not more than 18.5 ppm (37 ng/day) of acceptable daily intake (ADI); wherein the amounts are weight percentages based on the total weight of the formulation.
E2 The varenicline formulation of embodiment E1, wherein the level of N-nitrosovarenicline (NNV) impurity remains about 2-fold below acceptable daily intake (ADI) under accelerated storage conditions of 25° C. at 60% relative humidity for 6 months.
E3 The varenicline formulation of embodiment E1, wherein the level of N-nitrosovarenicline (NNV) impurity remains about 2-fold below acceptable daily intake (ADI) under accelerated storage conditions of 30° C. at 75% relative humidity for 6 months.
E4 The varenicline formulation of embodiment E1, wherein the level of N-nitrosovarenicline (NNV) impurity remains about 2-fold below acceptable daily intake (ADI) under accelerated storage conditions of 40° C. at 75% relative humidity for 6 months.
E5 ‘The varenicline formulation of embodiment E1, wherein the formulation prevents further N-nitrosovarenicline (NNV) impurity formation after three months.
E6 The varenicline formulation of any one of embodiments E1-E5, wherein the formulation contains about 2.0% to about 3.0% propyl gallate.
E7 The varenicline formulation of embodiment E6, wherein the formulation contains about 2.5% to about 3.0% propyl gallate.
E8 The varenicline formulation of embodiment E6, wherein the formulation contains about 2.57% propyl gallate.
E9 The varenicline formulation according to any one embodiments E1-E8, wherein the formulation does not contain silicon dioxide.
E10 In another embodiment of the invention there is provided a varenicline formulation comprising:
   a tablet core comprising:
   (a) about 0.85% varenicline tartrate;
   (b) about 60.50% microcrystalline cellulose;
   (c) about 33.33% dibasic calcium phosphate anhydrous;
   (d) about 2.00% croscarmellose sodium;
   (e) about 2.57% propyl gallate;
   (f) about 0.75% magnesium stearate;
   wherein the amounts are weight percentages based on the total weight of the tablet core;
   and
   a film coating, provided that the formulation does not contain silicon dioxide.
E11 The varenicline formulation of embodiment E10, wherein the formulation maintains a level of N-nitrosovarenicline (NNV) impurity over the shelf-life of the formulation of not more than 18.5 ppm (37 ng/day) of acceptable daily intake (ADI).
E12 The varenicline formulation of embodiment E11, wherein the level of N-nitrosovarenicline (NNV) impurity remains about 2-fold below acceptable daily intake (ADI) under accelerated storage conditions of 25° C. at 60% relative humidity for 6 months.
E13 The varenicline formulation of embodiment E11, wherein the level of N-nitrosovarenicline (NNV) impurity remains about 2-fold below acceptable daily intake (ADI) under accelerated storage conditions of 30° C. at 75% relative humidity for 6 months.
E14 The varenicline formulation of embodiment E11, wherein the level of N-nitrosovarenicline (NNV) impurity remains about 2-fold below acceptable daily intake (ADI) under accelerated storage conditions of 40° C. at 75% relative humidity for 6 months.
E15 The varenicline formulation of any one of embodiments E10-E14, wherein the formulation prevents further N-nitrosovarenicline (NNV) impurity formation after three months.
E16 The varencilcine formulation of any one of embodiments E10-E15, wherein the varenicline formulation contains approximately 0.5 mg varenicline tartrate per tablet.
E17 The varencilcine formulation of any one of embodiments E10-E15, wherein the varenicline formulation contains approximately 1.0 mg varenicline tartrate per tablet.
E18 In another embodiment of the invention there there is provided a varenicline formulation consisting of:
   a tablet core consisting of:
   (a) about 0.85% varenicline tartrate;
   (b) about 60.50% microcrystalline cellulose;
   (c) about 33.33% dibasic calcium phosphate anhydrous;
   (d) about 2.00% croscarmellose sodium;
   (e) about 2.57% propyl gallate;
   (f) about 0.75% magnesium stearate,
   wherein the amounts are weight percentages based on the total weight of the tablet core; and
   a film coating.
E19 The varenicline formulation of embodiment E18, wherein the formulation maintains a level of N-nitrosovarenicline (NNV) impurity over the shelf-life of the formulation of not more than 18.5 ppm (37 ng/day) of acceptable daily intake (ADI).
E20 The varenicline formulation of embodiment E19, wherein the level of N-nitrosovarenicline (NNV) impurity remains about 2-fold below acceptable daily intake (ADI) under accelerated storage conditions of 25° C. at 60% relative humidity for 6 months.
E21 The varenicline formulation of embodiment E19, wherein the level of N-nitrosovarenicline (NNV) impurity remains about 2-fold below acceptable daily intake (ADI) under accelerated storage conditions of 30° C. at 75% relative humidity for 6 months.
E22 The varenicline formulation of embodiment E19, wherein the level of N-nitrosovarenicline (NNV) impurity remains about 2-fold below acceptable daily intake (ADI) under accelerated storage conditions of 40° C. at 75% relative humidity for 6 months.
E23 The varenicline formulation of any one of embodiments E18-E22, wherein the formulation prevents further N-nitrosovarenicline (NNV) impurity formation after three months.
E24 The varencilcine formulation of any one of embodiments E18-E23, wherein the varenicline formulation contains approximately 0.5 mg varenicline tartrate per tablet.
E25 The varencilcine formulation of any one of embodiments E18-E23, wherein the varenicline formulation contains approximately 1.0 mg varenicline tartrate per tablet.
E26 A method for reducing nicotine addiction or aiding in the cessation or lessening of tobacco use in a mammal, comprising administering to said mammal atherapeutically effective amount of a formulation according to any one of embodiments E1-25 that is effective in reducing nicotine addiction or aiding in the cessation of lessening of tobacco use.
E27 The varenicline formulation of any one of embodiments E1-E25, wherein the formulation maintains a level of N-nitrosovarenicline (NNV) impurity over the shelf-life of the formulation from about 3 ppm to about 18.5 ppm of acceptable daily intake (ADI).
E28 The varenicline formulation of any one of embodiments E1-E25, wherein the formulation maintains a level of N-nitrosovarenicline (NNV) impurity over the shelf-life of the formulation from about 4 ppm to about 15 ppm of acceptable daily intake (ADI).
E29 The varenicline formulation of any one of embodiments E1-E25, wherein the formulation maintains a level of N-nitrosovarenicline (NNV) impurity over the shelf-life of the formulation from about 5 ppm to about 10 ppm of acceptable daily intake (ADI).
E30 The varenicline formulation of any one of embodiments E1-E25, wherein the formulation maintains a level of N-nitrosovarenicline (NNV) impurity over the shelf-life of the formulation from about 6 ppm to about 9 ppm of acceptable daily intake (ADI).
E31 The varenicline formulation of any one of embodiments E1-E25, wherein the formulation maintains a level of N-nitrosovarenicline (NNV) impurity over the shelf-life of the formulation of not more than 10 ppm of acceptable daily intake (ADI).
E32 The varenicline formulation of any one of embodiments E1-E25, wherein the formulation maintains a level of N-nitrosovarenicline (NNV) impurity over the shelf-life of the formulation of not more than 9 ppm of acceptable daily intake (ADI).
E33 The varenicline formulation of any one of embodiments E1-E25, wherein the formulation maintains a level of N-nitrosovarenicline (NNV) impurity over the shelf-life of the formulation of not more than 6 ppm of acceptable daily intake (ADI).
E34 The varenicline formulation of any one of embodiments E1-E25, wherein the formulation maintains a level of N-nitrosovarenicline (NNV) impurity over the shelf-life of the formulation of not more than 3 ppm of acceptable daily intake (ADI).

### Definitions

Unless otherwise defined herein, scientific and technical terms used in connection with the present invention have the meanings that are commonly understood by those of ordinary skill in the art. The invention described herein suitably may be practiced in the absence of any element(s) not specifically disclosed herein.

In this specification and in the claims that follow, reference will be made to a number of terms that shall be defined to have the following meanings:

As used herein, the singular form “a”, “an”, and “the” include plural references unless indicated otherwise. For example, “a” substituent includes one or more substituents.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. The expression “prodrug” refers to compounds that are drug precursors which following administration, release the drug in vivo via some chemical or physiological process (e.g., a prodrug on being brought to the physiological pH or through enzyme action is converted to the desired drug form).

The term “nitrosamine” describes a class of compounds having the chemical structure of a nitroso group bonded to an amine (R₁N(—R₂)—N═O). The compounds can form by a nitrosating reaction between amines (secondary, tertiary, or quaternary amines) and nitrous acid (nitrite salts under acidic conditions). Examples of nitrosamines include, but are not limited to N-nitrosodimethylamine (NDMA), N-nitrosodiethylamine (NDEA), N-nitroso-N-methyl-4-aminobutanoic acid (NMBA), N-nitrosoisopropylethyl amine (NIPEA), N-nitrosodiisopropylamine (NDIPA), N-nitrosodibutylamine (NDBA), and N-nitrosomethylphenylamine (NMPA).

The term “free of nitrosamine” or “nitrosamine free” means that there is no detectable level of nitrosamine impurities in the drug product.

The average intake (AI) limit is a daily exposure to a compound such as NDMA, NDEA, NMBA, NMPA, NIPEA, or NDIPA that approximates a 1:100,000 cancer risk after 70 years of exposure. The conversion of AI limit into ppm varies by product and is calculated based on a drug's maximum daily dose (MDD) as reflected in the drug label (ppm=AI (ng)/MDD (mg)).

“Patient” refers to warm blooded animals such as, for example, guinea pigs, mice, rats, gerbils, cats, rabbits, dogs, cattle, goats, sheep, horses, monkeys, chimpanzees, and humans.

“Therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

### EXPERIMENTALS

The following illustrate the synthesis of various compounds of the present invention. Additional compounds within the scope of this invention may be prepared using the methods illustrated in these Examples, either alone or in combination with techniques generally known in the art. All starting materials in these Preparations and Examples are either commercially available or can be prepared by methods known in the art or as described herein.

### Example 1

U.S. Pat. No. 6,410,550, the disclosure of which is hereby incorporated by reference in its entirety, describes aryl-fuse azapolycyclic compounds, methods for synthesis and their use in various disease states including reducing nicotine addiction or aiding in the cessation or lessening of tobacco use. Example 26 of the '550 patent is specific to the compound 5,8,14-triazatetracyclo[10.3.1.0^{2,11}.0^{4,9}]hexadeca-2(11),3,5,7,9-pentaene hydrochloride (varenicline)

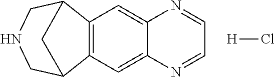

that is synthesized by the following steps:

### A) 1-(4,5-Diamino-10-aza-tricyclo[6.3.1.0^{2,7}]dodeca-2(7),3,5-trien-10-yl)-2,2,2-trifluoro-ethanone

1-(4,5-Dinitro-10-aza-tricyclo[6.3.1.0^{2,7}]dodeca-2(7),3,5-trien-10-yl)-2,2,2-trifluoro-ethanone (3.0 g, 8.70 mmol) was hydrogenated in MeOH (30 ml) under H₂ (45 psi) over Pd(OH)₂ (300 mg of 20 wt %/C, 10% wt). After 2.5 hours the reaction was filtered through a Celite pad and rinsed with MeOH (30 ml). The solution was concentrated to a light brown oil which crystallized (2.42 g, 96%). (TLC 10% MeOH/CH₂Cl₂ R_{f} 0.56). APCI MS m/e 286.2 [(M+1)⁺]. mp 129-131° C.

### B) 1-(5,8,14-Triazatetracyclo[10.3.1.0^{2,11}.0^{4,9}]hexadeca-2(11),3,5,7,9-pentaene)-2.2.2-trifluoro-ethanone

1-(4,5-Diamino-10-aza-tricyclo[6.3.1.0^{2,7}]dodeca-2(7),3,5-trien-10-yl)-2,2,2-trifluoro-ethanone (500 mg, 1.75 mmol) was stirred in THF (2 ml). This mixture was treated with H₂O (2 mL) and glyoxal sodium bisulfite addition compound hydrate (931 mg, 3.50 mmol) then stirred at 55° C. for 2.5 hours. The reaction was cooled to room temperature and extracted with EtOAc (3×40 ml). The combined organic layer was washed with H₂O (2×30 ml), dried (Na₂SO₄), filtered, concentrated and chromatographed on Silica gel to provide an off white powder (329 mg, 60%). (TLC 25% EtOAc/hexanes R_{f} 0.40). mp 164-166° C.

### C) 5,8,14-Triazatetracyclo[10.3.1.0^{2,11}.0^{4,9}]hexadeca-2(11),3,5,7,9-pentaene hydrochloride

1-(5,8,14-Triazatetracyclo[10.3.1.0^{2,11}.0^{4,9}]hexadeca-2(11),3,5,7,9-pentaene)-2,2,2-trifluoro-ethanone (320 mg, 1.04 mmol) was slurried in MeOH (2.0 ml) and treated with Na₂CO₃ (221 mg, 2.08 mmol) in H₂O (2.0 ml). The mixture was warmed to 70° C. for 2 hours, then concentrated, treated with H₂O (20 mL) and extracted with CH₂Cl₂ (3×10 ml). The organic layer was dried through a cotton plug and concentrated to give a light yellow oil (183 mg, 83%) which solidified upon standing (mp 138-140° C.). This material was dissolved in MeOH (10 mL), treated with 3M HCl/EtOAc (3 ml), concentrated and azeotroped with MeOH (2×20 mL) to give solids which were recrystallized from MeOH/Et₂O to afford product as a white solid (208 mg, 97%). (TLC 5% MeOH/CH₂Cl₂ (NH₃) R_{f} 0.26). 1H NMR (400 MHz, CD₃OD) δ8.94 (s, 2H), 8.12 (s, 2H), 3.70 (m, 2H), 3.54 (d, J=12.5 Hz, 2H), 3.35 (d, J=12.5 Hz, 2H), 2.49 (m, 1H), 2.08 (d, J=11.0 Hz, 1H). GCMS m/e 211 (M+). mp 225-230° C.

### Example 2

U.S. Pat. Nos. 6,890,927 and 7,265,119, the disclosures of which is hereby incorporated by reference in its entirety, describe polymorphic and salt forms of 5,8,14-triazatetracyclo [10.3.1.0^{2,11}.0^{4,9}]hexadeca-2(11),3,5,7,9-pentaene (varenicline), including the commercial tartrate salt form that was approved by the U.S. FDA and various other world regulatory agencies as an aid to smoking cessation treatment.

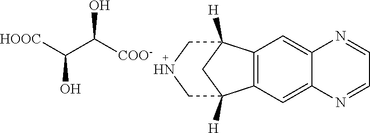

### Examples 3 and 4

### Preparation of 0.5 mg (Example 3) and 1.0 mg (Example 4) Varenicline Tartrate Tablets (Chantix®)

Varenicline tartrate tablets, 0.5 and 1.0 mg were manufactured by a standard manufacturing process that includes blending, milling (de-agglomeration), lubricant blending, roller compacting, milling, blending, lubricant blending, compressing, and film-coating processes using equipment commonly available in the pharmaceutical industry. In addition, containment and automation technologies were implemented throughout the manufacturing process to facilitate the manufacture of varenicline tartrate tablets, 0.5 and 1.0 mg. Formulation compositions for the 0.5 mg and 1.0 mg varencilcine tartrate tablets are provided in Table 1:

TABLE 1
Varenicline Tartrate 0.5 mg and 1.0 mg Tablets

| | 0.5 mg Varenicline | | 1.0 mg Varenicline | |
|---|---|---|---|---|
| | Tartrate Tablets | | Tartrate Tablets | |
| | (Chantix ®) | | (Chantix ®) | |
| | Formulation | | Formulation | |
| | (Example 3) | | (Example 4) | |
| | Quantity/tablet | as % | Quantity/tablet | as % |
| Component | (mg) | w/w | (mg) | w/w |

Core Tablet

| | | | | |
|---|---|---|---|---|
| Varenicline | 0.85 | 0.855 | 1.71 | 0.855 |
| Tartrate^{a} | | | | |
| Microcrystalline | 62.57 | 62.565 | 125.13 | 62.565 |
| Cellulose^{b} | | | | |
| Dibasic Calcium | 33.33 | 33.330 | 66.66 | 33.330 |
| Phosphate, | | | | |
| anhydrous | | | | |
| Croscarmellose | 2.00 | 2.000 | 4.00 | 2.000 |
| Sodium | | | | |
| Colloidal | 0.50 | 0.500 | 1.00 | 0.500 |
| Silicon Dioxide | | | | |
| Magnesium | 0.75 | 0.750 | 1.5 | 0.750 |
| Stearate | | | | |
| Core Table | 100.00 | 100.000 | 200.00 | 100.000 |
| Weight | | | | |

Film Coat Solution

| | | | | |
|---|---|---|---|---|
| Opadry ® White | 4.00 | 4.000 | 8.00 | 4.000 |
| (YS-1-182002-A)/ | | | | |
| Opadry ® Blue | | | | |
| (03B90547) | | | | |
| Purified Water^{c} | 22.67 | — | 43.340 | — |
| Opadry ® Clear | 0.5 | 0.500 | 1.0 | 0.500 |
| (YS-2-19114-A) | | | | |
| Purified Water^{c} | 9.50 | — | 19.00 | — |
| Total Film Coated | 104.50 | 104.500 | 209.00 | 104.500 |
| Tablet Weight | | | | |

^{a}Based on theoretical potency of 58.5%
^{b}Weight my be adjusted for slight potency differences in varenicline tartrate
^{c}Volatile, removed during process

The 0.5 mg and 1.0 mg varencilcine tartrate tablets were manufactured utilizing the following process steps:
Step 1—Milling/Blending (pre-blend): a first part of microcrystalline cellulose and varenicline tartrate were loaded, alternatively, in small portions, into the milling chamber, passed through the mill and charged into a bin. The sequence was repeated until total usage of the microcrystalline cellulose and varenicline. The remaining part of microcrystalline cellulose, anhydrous dibasic calcium phosphate, croscarmellose sodium, and colloidal silica anhydrous were passed through the mill, charged into a bin and tumbled.
Step 2—Lubricating/Blending (Initial): the magnesium stearate is sieved, and approximately two-thirds of the total amount of magnesium stearate is charged into the blend from Step 1. The contents were then tumbled.
Step 3—Granulation: the blend from Step 2 is dry granulated using a dry granulator (Roll Force-Bepex: 40-60 kN, Gerteis 5-9 kN; Gap Width-Gerteis 1.7-3.5 mm).
Step 4—Milling: the compacted blend from Step 3 is milled using a screening mill fitted with a screen using sufficient mill speed (Mill Screen Size-Bepex: 0.8-1.0 mm, Gerteis 0.8-1.5 mm).
Step 5—Blending: the granulation from Step 4 is charged into a suitable bin and blended.
Step 6—Lubricating/Blending (Final): the remaining sieved magnesium stearate is charged to the blend from Step 5 and blended.
Step 7—Tabletting (Cores): the blend from Step 6 is compressed on a tablet press. The tablet press was shut off with not less than 0.5 kg blend remaining.
Step 8—Preparation of Film Coating Suspension I: For the 0.5 mg varenicline tartrate tablets, the film coating suspension was prepared using Opadry® White (YS-1-18202-A) and Purified Water. For the 1.0 mg varenicline tartrate tablets, the film coating suspension was prepared using Opadry® Blue (03B90547) and Purified Water. Both coating suspensions were prepared in excess to cover expected losses during the manufacturing process.
Step 9—Preparation of Film Coating Suspension II: the film coating suspension was prepared using Opadry® Clear (YS-2-19114-A) and Purified Water. The suspension was prepared in excess to cover expected losses during the manufacturing process.
Step 10—Film Coating: the 0.5 mg and 1.0 mg tablet cores prepared in Step 7 were film coated with the respective Film Coating Suspension I prepared in Step 8. The tablets film coated with Suspension I were then film coated with Film Coating Suspension II prepared in Step 9 to obtain the final 0.5 mg and 1.0 mg tablets, respectively.

### Examples 5 and 6

### Preparation of 0.5 mg (Example 5) and 1.0 mg (Example 6) Varenicline Tartrate Propyl Gallate (PG) Tablets

Varenicline tartrate PG tablets, 0.5 and 1.0 mg were manufactured by a standard manufacturing process that includes blending, milling (de-agglomeration), lubricant blending, roller compacting, milling, blending, lubricant blending, compressing, and film-coating processes using equipment commonly available in the pharmaceutical industry. In addition, containment and automation technologies were implemented throughout the manufacturing process to facilitate the manufacture of varenicline tartrate tablets, 0.5 and 1.0 mg. Formulation compositions for the 0.5 mg and 1.0 mg varencilcine tartrate PG tablets are provided in Table 2:

TABLE 2
Varenicline Tartrate 0.5 mg and 1.0
mg Propyl Gallate (PG) Tablets

| | 0.5 mg Varenicline | | 1.0 mg Varenicline | |
|---|---|---|---|---|
| | Tartrate PG | | Tartrate PG | |
| | Tablet Formulation | | Tablet Formulation | |
| | (Example 5) | | (Example 6) | |
| | Quantity/tablet | as % | Quantity/tablet | as % |
| Component | (mg) | w/w | (mg) | w/w |

Core Tablet

| | | | | |
|---|---|---|---|---|
| Varenicline | 0.85 | 0.855 | 1.71 | 0.855 |
| Tartrate^{a} | | | | |
| Microcrystalline | 60.50 | 60.500 | 121.00 | 60.500 |
| Cellulose^{b} | | | | |
| Dibasic Calcium | 33.33 | 33.330 | 66.66 | 33.330 |
| Phosphate, | | | | |
| anhydrous | | | | |
| Croscarmellose | 2.00 | 2.000 | 4.00 | 2.000 |
| Sodium | | | | |
| Colloidal | — | — | — | — |
| Silicon Dioxide | | | | |
| Propyl Gallate | 2.57 | 2.565 | 5.13 | 2.565 |
| Magnesium | 0.75 | 0.750 | 1.5 | 0.750 |
| Stearate | | | | |
| Core Table | 100.00 | 100.000 | 200.00 | 100.000 |
| Weight | | | | |

Film Coat Solution

| | | | | |
|---|---|---|---|---|
| Opadry ® White | 4.00 | 4.000 | 8.00 | 4.000 |
| (YS-1-182002-A)/ | | | | |
| Opadry ® Blue | | | | |
| (03B90547) | | | | |
| Purified Water^{c} | 22.67 | — | 43.340 | — |
| Opadry ® Clear | 0.5 | 0.500 | 1.0 | 0.500 |
| (YS-2-19114-A) | | | | |
| Purified Water^{c} | 9.50 | — | 19.00 | — |
| Total Film Coated | 104.50 | 104.500 | 209.00 | 104.500 |
| Tablet Weight | | | | |

^{a}Based on theoretical potency of 58.5%
^{b}Weight my be adjusted for slight potency differences in varenicline tartrate
^{c}Volatile, removed during process

The 0.5 mg and 1.0 mg varencilcine tartrate PG tablets were manufactured utilizing the following process steps:
Step 1—Milling/Blending (pre-blend): microcrystalline cellulose, varenicline tartrate, and propyl gallate were loaded alternatively into the milling chamber, passed through the mill and charged into a bin. The sequence was repeated until total usage of the microcrystalline cellulose varenicline, and propyl gallate. Croscarmellose sodium, and anhydrous dibasic calcium phosphate were passed through the mill, charged into a bin and tumbled.
Step 2—Lubricating/Blending (Initial): the magnesium stearate is sieved, and approximately two-thirds of the total amount of magnesium stearate is charged into the blend from Step 1. The contents were then tumbled.
Step 3—Granulation: the blend from Step 2 is dry granulated using a dry granulator (Roll Force—Bepex: 40-60 kN).
Step 4—Milling: the compacted blend from Step 3 is milled using a screening mill fitted with a screen using sufficient mill speed (Mill Screen Size—Bepex: 0.8-1.0 mm).
Step 5—Blending: the granulation from Step 4 is charged into a suitable bin and blended.
Step 6—Lubricating/Blending (Final): the remaining sieved magnesium stearate is charged to the blend from Step 5 and blended.
Step 7—Tabletting (Cores): the blend from Step 6 is compressed on a tablet press. The tablet press was shut off with not less than 0.5 kg blend remaining.
Step 8—Preparation of Film Coating Suspension I: For the 0.5 mg varenicline tartrate PG tablets, the film coating suspension was prepared using Opadry® White (YS-1-18202-A) and Purified Water. For the 1.0 mg varenicline tartrate PG tablets, the film coating suspension was prepared using Opadry® Blue (03B90547) and Purified Water. Both coating suspensions were prepared in excess to cover expected losses during the manufacturing process.
Step 9—Preparation of Film Coating Suspension II: the film coating suspension was prepared using Opadry® Clear (YS-2-19114-A) and Purified Water. The suspension was prepared in excess to cover expected losses during the manufacturing process.
Step 10—Film Coating: the 0.5 mg and 1.0 mg PG tablet cores prepared in Step 7 were film coated with the respective Film Coating Suspension I prepared in Step 8. The tablets film coated with Suspension I were then film coated with Film Coating Suspension II prepared in Step 9 to obtain the final 0.5 mg and 1.0 mg tablets, respectively.

### Example 7

### Evaluation of Nitrite Content in Batches of Microcrystalline Cellulose (MCC)

Microcrystalline cellulose (MCC) batches from alternative suppliers were evaluated to determine if changing the supplier of MCC could reduce the NNV to below the requested Acceptable Daily Intake (ADI) of not more than (NMT) 37 ng/day. Differences in the levels of nitrite were observed between suppliers and within batches provided by the same supplier. Batches of MCC were evaluated for nitrite content and found to contain up to 3.5 ppm (Table 3).

TABLE 3
Nitrite levels measured in batches of MCC

| Batch | Nitrite Level (ppm) |
|---|---|
| A | 0.4 |
| B | 1.0 |
| C | 0.8 |
| D | 1.8 |
| E | 0.8 |
| F | 2.9 |
| G | 1.7 |
| H | 3.5 |

### Example 8

### Root Cause Analysis of N-Nitrosovarenicline Formation

A root cause analysis investigation to determine the causes of N-nitrosovarenicline (NNV) formation in the currently approved varenicline tartrate tablets (Chantix®) was performed.

Ternary compacts containing varenicline tartrate (API), microcrystalline cellulose (MCC; 3 ppm nitrite) and minor components colloidal silicon dioxide (SiO₂), croscarmellose sodium (CCS), magnesium stearate (Mg St) or Opadry blue (Opadry) were prepared at lab scale and placed in a closed glass container and held at 60° C. to accelerate the formation of NNV. Testing for NNV was performed after 7 days. The batches evaluated are described in Table 4:

TABLE 4
NNV levels measured in compacts containing
API:MCC with minor components in Chantix ® tablet
formulation after storage

| | | Mean NNV (ppm) after |
|---|---|---|
| Batch | Sample Compact | 7 days at 60° C. |
| A | API:MCC | 455 |
| | (control) | (465, 445) |
| B | API:MCC:SiO₂ | 1157 |
| | | (1180, 1133) |
| C | API:MCC:CCS | 522 |
| | | (524, 519) |
| D | API:MCC:MgSt | 1082 |
| | | (1079, 1084) |
| E | API:MCC:Opadry | 415 |
| | | (430, 400_{—} |

Approximately four (4) tablets were set down in closed glass containers for each batch, temperature and timepoint. Two individual tablets were tested for NNV.

The data shows that the primary contributor to the formation of NNV was identified to be the presence of nitrite species in microcrystalline cellulose (MCC), the major component in the formulation, which reacts with the secondary amine present in the varenicline molecule to form NNV.

The data further shows that croscarmellose sodium and Opadry do not have a significant impact on NNV formation in combination with MCC. However, Magnesium stearate in combination with MCC did demonstrate a significant increase in NNV formation. It was determined that the removal of magnesium stearate was not considered viable, since it is used as the lubricant and is needed to avoid sticking during roller compaction and tablet compression. In contrast, and unexpectedly, it was also discovered that even a minute amount of colloidal silicone dioxide in the compact significantly increased the level of NNV. Since contemporary tablet formulations with similar diluent core components do not typically include a glidant such as colloidal silicon dioxide, it was decided to remove colloidal silicon dioxide in future reformulated tablets to understand what impact, if any, this would have on mitigating for the formation of NNV.

### Example 9

Experiments demonstrated, even when using MCC batches with a nitrite level of ≤0.1 ppm and removing CSD, that it was not possible to consistently control the NNV levels to below 37 ng/day using the currently approved varenicline tartrate tablet formulation. Therefore, a further decision was made to evaluate if the addition of an antioxidant to the varenicline tartrate (Chantix®) formulation would have an effect on the formation of NNV.

### Example 10

### Summary of the Assessment of Antioxidants in Varenicline Cores

A screening study was performed to determine if the addition of commonly used antioxidants to a representative Chantix® core formulation, could limit the formation of N-nitroso varenicline (NNV) to less than 18.5 ppm.

Three antioxidants, ascorbic acid, citric acid and propyl gallate, were initially evaluated to determine their potential suitability for controlling NNV formation in varenicline tartrate tablets to below the requested ADI. The formulations were prepared at laboratory scale as uncoated direct compressed tablet cores and were composed of the same formulation as the currently approved 1.0 mg varenicline tartrate (Chantix®) tablets, with the amount of MCC present adjusted to account for the addition of 1% or 3% antioxidant respectively. The formulations were made with MCC containing 3 ppm nitrite which was at the high end of the range found in MCC batches previously tested for nitrite (See Example 7, Table 3). Samples of these formulations were placed in a closed glass container for 7 days at 60° C. to accelerate NNV formation. The results of the screening test are provided in Table 5 below:

TABLE 5

NNV (ppm)

| | | 7 days at |
|---|---|---|
| Antioxidant | Initial | 60° C. |
| 1% Ascorbic acid | 2 | 397 |
| 3% Ascorbic acid | 3 | 288 |
| 1% Citric acid | 6 | 684 |
| 3% Citric acid | 7 | 691 |
| 1% Propyl gallate | 14 | 20 |
| 3% Propyl gallate | 6 | 11 |

The study demonstrated that tablets with propyl gallate (PG) at an initial screening concentration of 3% w/w achieved NNV levels below the requested target of 18.5 ppm (equivalent to requested ADI of NMT 37 ng/day).

Based on the results of the study, it was determined that a propyl gallate level in the region of 3% w/w core was required, taking into consideration potential nitrite levels up to 3.5 ppm in the MCC and the desire to robustly control NNV levels below the requested ADI.

### Example 11

### Manufacture of Varenicline Propyl Gallate Formulations

Using the understanding of propyl gallate level needed to reduce NNV formation as described in Example 8 and Example 10, two batches of the 1.0 mg varenicline tartrate PG tablets were manufactured to determine if NNV levels below the ADI could be replicated at a representative commercial scale (115 kg).

One batch was manufactured using propyl gallate added at 3 times the weight of the API and was close to the concentration used at the small scale (2.57% w/w core: 5.13 mg/tablet). The second batch was manufactured using propyl gallate added at 5 times the weight of the API (4.28% w/w core: 8.55 mg/tablet). The higher level of propyl gallate of 5×API concentration was chosen to determine if additional propyl gallate may be needed at the commercial scale.

The process parameters used were the same as for the current commercial drug product and the 1.0 mg dosage strength was selected as representative of the formulation since the 0.5 mg and 1.0 mg formulation cores are a common blend. The nitrite content of the MCC was 1.7 ppm (approximately 50% of the highest level observed in evaluated MCC batches).

Results from the tests performed for varenicline tartrate tablets PG, batches FR2855 (2.57% w/w core PG) and FR2856 (4.28% w/w core PG), are provided in Table 6.

TABLE 6

| | Batch A | Batch B |
|---|---|---|
| | (2.57% w/w | (4.28% w/w |
| Batch number | propyl gallate) | propyl gallate) |
| Assay¹ | 100.1% LC | 99.3% LC |
| CP-697,535 | NMT 0.05% | NMT 0.05% |
| CP-536,363 | NMT 0.05% | NMT 0.05% |
| Individual impurities | NMT 0.05% | NMT 0.05% |
| Total impurities | NMT 0.05% | NMT 0.05% |
| Propyl gallate | 98.9% | 99.4% |
| Disintegration (mean)² | 1.3 minutes | 1.3 minutes |
| N-nitrosovarenicline (NNV)³ | 4.4 ppm | 4.0 ppm |

¹Currently approved criterion 90.0-110.0%
²Currently approved criterion NMT 10 minutes
³Proposed acceptance criterion NMT 18.5 ppm
LC = Label claim;
NMT = Not More Than

Assay, impurities, and disintegration were determined using the varenicline tartrate tablet test methods registered at the time of testing. The levels of propyl gallate and NNV were assessed with suitably qualified methods.

The results confirm that the addition of propyl gallate and the removal of colloidal silicon dioxide, along with microcrystalline cellulose adjustment, have had no significant impact on the quality, purity, or performance of the drug product.

### Example 12

### Assessment of NNV and Propyl Gallate Stability in Varenicline Tartrate PG Tablets

As demonstrated by the antioxidant screening study described in Example 10, the formation of NNV can be accelerated by exposing the drug product to high temperatures such as 60° C. Therefore, to generate more confidence in the ability of propyl gallate to inhibit the formation of NNV long term and to select the minimum level of PG needed to maintain the NNV level below the ADI, an accelerated stability study with different humidity conditions over multiple time points was conducted on the representative scale (115 kg) batches.

The NNV results from the accelerated stability at 60° C. for Batch A and Batch B are provided in Tables 7-9.

TABLE 7
Accelerated Stability (at 60° C.) NNV
Data for Batch A (2.57% w/w propyl gallate)

| | NNV ppm¹ | | | | |
|---|---|---|---|---|---|
| Condition | Initial | 3 Days | 7 Days | 14 Days | 28 Days |
| 60° C./75% RH | 4.4 | 6.2 | 5.3 | 6.2 | 6.0 |
| 60° C./50% RH | | 6.3 | 6.5 | 5.5 | 7.7 |
| 60° C./natural RH | | 6.3 | 6.0 | 6.8 | 6.7 |
| 60° C./Desc | | 5.9 | 6.0 | 5.7 | 5.6 |
| Mean | 4.4 | 6.2 | 6.0 | 6.1 | 6.5 |

Natural = unadjusted humidity;
Desc = 1 g desiccant added
¹Mean of 2 results from individual tablets

TABLE 8
Accelerated Stability (at 60° C.) NNV
Data for Batch B (4.28% w/w propyl gallate)

| | NNV ppm¹ | | | | |
|---|---|---|---|---|---|
| Condition | Initial | 3 Days | 7 Days | 14 Days | 28 Days |
| 60° C./75% RH | 4.0 | 4.9 | 4.5 | 3.7 | 5.5 |
| 60° C./50% RH | | 5.1 | 5.1 | 4.8 | 5.5 |
| 60° C./natural RH | | 5.0 | 4.6 | 4.1 | 6.0 |
| 60° C./Desc | | 4.6 | 4.9 | 4.2 | 4.0 |
| Mean | 4.0 | 4.9 | 4.8 | 4.4 | 5.3 |

Natural = unadjusted humidity;
Desc = 1 g desiccant added
¹Mean of 2 results from individual tablets

The results show that NNV levels increase slightly on stability from the initial levels measured during release testing. However, after 3 days at 60° C. storage the levels plateau and no further significant increases in NNV level were observed out to 28 days. Additionally, there was no significant trend in NNV levels with different humidity conditions supporting that specific control of water content (e.g. via packaging) would not be necessary to control NNV levels.

Batch B which contained the higher level of PG, had a lower plateau (NNV mean 4.4-5.3 ppm) compared to Batch A which contained the lower level of PG (NNV mean 6.0-6.5 ppm). The NNV plateau levels observed in these batches, which had an ingoing MCC nitrite content of 1.7 ppm (approximately 50% of the maximum observed), represent the equivalent of 24-29% and 32-35% of the requested ADI respectively.

Accelerated data (60° C.) for propyl gallate in Batches A and B is presented in Table 9.

TABLE 9
Propyl Gallate Levels Observed During Accelerateed Stability
at 60° C. for Batches A (FR2855) and B (FR2856)

| | Propyl Gallate as % Label Claim | |
|---|---|---|
| | FR2855 (2.57% w/w | FR2856 (4.28% w/w |
| Conditions | propyl gallate) | propyl gallate) |
| Initial | 98.9 | 99.4 |
| 7 D 60° C./75% RH | 96.0 | 95.1 |
| 14 D 60° C./75% RH | 92.6 | 92.3 |
| 28 D 60° C./75% RH | 89.6 | 90.9 |

The data shows that propyl gallate levels decrease on stability at a similar rate in Batch A and Batch B with up to 10% loss of propyl gallate after 28 days at 60° C./75% RH. Despite the loss of up to 10% propyl gallate, NNV levels remained at their plateau levels. Therefore, since both batches had plateau NNV levels greater than 2-fold below the ADI and similar rate of loss of PG, it was decided to progress Batch A (2.57% w/w core PG) which had the lower level of PG, into a packaged stability study to determine if the NNV and PG levels observed in the accelerated studies at 60° C. were representative of what would be observed at accelerated (40° C.) and long-term storage conditions.

### Example 13

### Packaged Stability Data for Batch A (2.57% w/w PG)

Stability studies on the representative batch FR2855 in HDPE bottles (56 count) at accelerated and long-term conditions were tested over six months.

Table 10 and Table 11 summarizes the NNV and propyl gallate data from the packaged stability study up to 6 months.

TABLE 10
NNV Data for Batch A (2.57% w/w
PG) Packaged in HDPE Bottles

NNY ppm¹

| Condition | Initial | 1 M | 2 M | 3 M | 6 M |
|---|---|---|---|---|---|
| 25° C./60% RH | 6.7 | 5.9 | 9.1 | 4.4 | 4.5 |
| 30° C./75% RH | | 6.1 | 8.7 | 4.0 | 4.8 |
| 40° C./75% RH | | 6.2 | 8.9 | 4.4 | 4.6 |

¹Mean of 2 results from individual tablets

TABLE 11
Propyl Gallate Data for Batch A (2.57%
w/w PG) Packaged in HDPE Bottles

Propyl Gallate as % Label Claim

| Condition | Initial | 1 M | 2 M | 3 M | 6 M |
|---|---|---|---|---|---|
| 25° C./60% RH | 99.8 | 98.9 | NT | 99.6 | 96.0 |
| 30° C./75% RH | | 94.6 | NT | 100.4 | 92.9 |
| 40° C./75% RH | | 97.0 | NT | 97.1 | 92.0 |

NT = Not Tested

The NNV results were found to be between 4.0 and 9.1 ppm. The highest levels (8.7-9.1 ppm; 47-49% ADI) in the packaged stability study were seen at the 2-month checkpoint for all conditions. However, at 3 to 6 months the levels were found to be similar in keeping with the observed plateauing observed in the accelerated studies. The levels of propyl gallate were found to decreased between 4-8% after 6 months also in keeping with the observed reductions in the accelerated studies at 60° C.

In conclusion, propyl gallate added at a level of 2.57% w/w core, has ensured that NNV levels remain approximately 2-fold below the ADI out to 6 months for a batch which contained MCC at 50% of the highest nitrite seen in the MCC batches. There is a plateauing trend for NNV levels from 3 months onwards demonstrating that the selected level of propyl gallate is preventing further NNV generation. Therefore, taking into consideration the need to accommodate the range of nitrite levels that were measured in MCC batches from the current supplier (up to 3.5 ppm) the selection of PG added at 2.57% w/w core will maintain NNV below the requested ADI across the current shelf-life whilst not including unnecessary amounts of PG in the formulation.

### Example 14

### Comparison of Drug-Release Profiles of Varenicline Tartrate 0.5 mg Propyl Gallate Tablets, 1.0 mg Propyl Gallate Tablets and Chantix® IR 0.5 mg and 1.0 mg Tablets

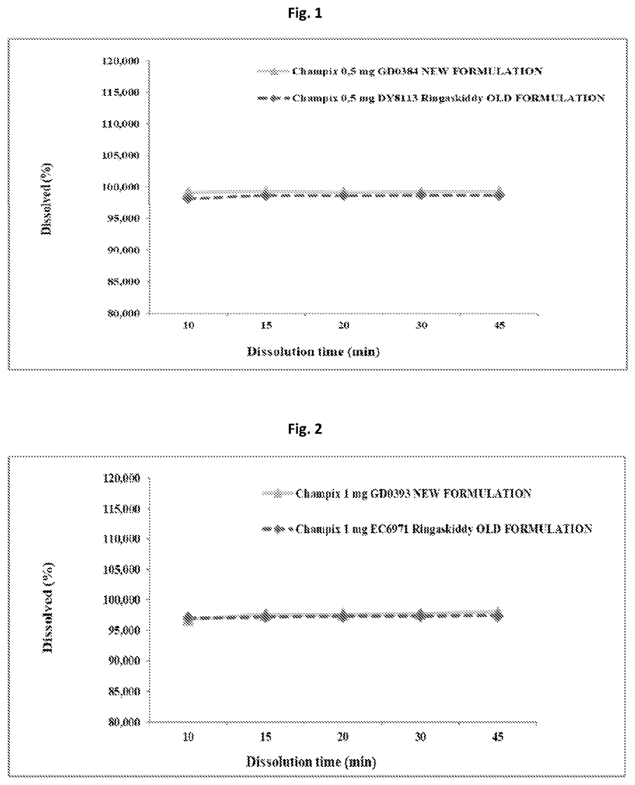

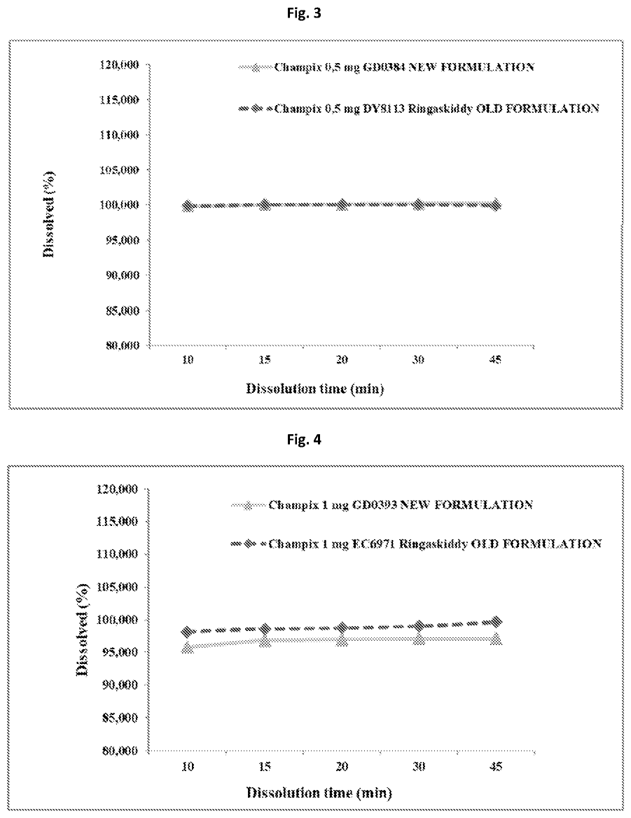

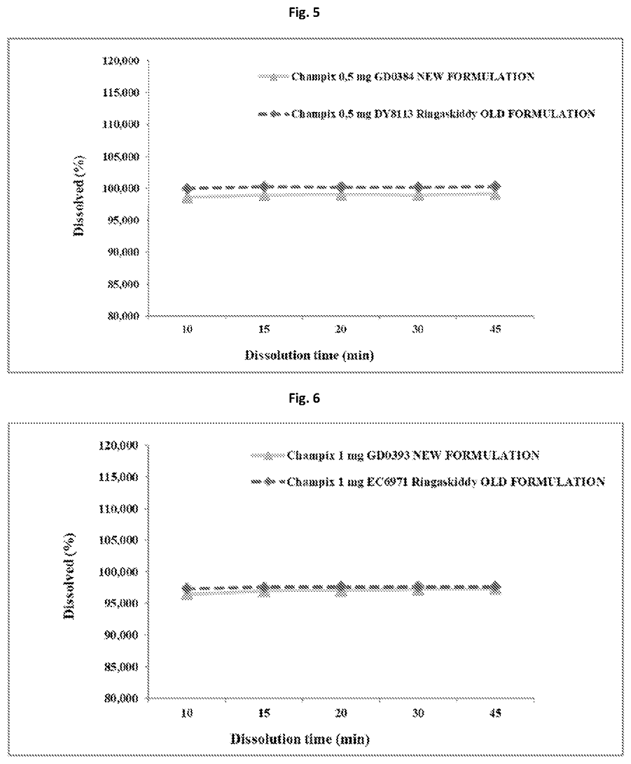

To demonstrate that the addition of PG, removal of CSD and adjustment of MCC has not impacted the performance of the drug product, in vitro dissolution tests have been conducted on varenicline tartrate 0.5 mg and 1.0 mg tablets PG (0.5 mg lot GD0384 and 1.0 mg lot GD0393), which are representative of the proposed commercial formulation and manufacturing process for the test product. These results have been compared to results generated for the reference product (CHANTIX® IR Tablets, 0.5 mg, commercial batch DY8113 and CHANTIX® IR Tablets, 1 mg, commercial batch EC6971). Multimedia dissolution data (performed using USP 1 apparatus (baskets), 100 rpm with 0.1N HCl, pH 4.5 acetate buffer and pH 6.8 phosphate buffer, all at 37° C.). The mean dissolution results in the different media for both dosage strengths are summarized in FIGS. 1-6.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-9. (canceled)

10. A varenicline formulation comprising:
a tablet core comprising:
(a) about 0.85% varenicline tartrate;
(b) about 60.50% microcrystalline cellulose;
(c) about 33.33% dibasic calcium phosphate anhydrous;
(d) about 2.00% croscarmellose sodium;
(e) about 2.57% propyl gallate;
(f) about 0.75% magnesium stearate;
wherein the amounts are weight percentages based on the total weight of the tablet core; and
a film coating, provided that the formulation does not contain silicon dioxide.

11. The varenicline formulation of claim 10, wherein the formulation maintains a level of N-nitrosovarenicline (NNV) impurity over the shelf-life of the formulation of not more than 18.5 ppm (37 ng/day) of acceptable daily intake (ADI).

12. The varenicline formulation of claim 11, wherein the level of N-nitrosovarenicline (NNV) impurity remains about 2-fold below acceptable daily intake (ADI) under accelerated storage conditions of 25° C. at 60% relative humidity for 6 months.

13. The varenicline formulation of claim 11, wherein the level of N-nitrosovarenicline (NNV) impurity remains about 2-fold below acceptable daily intake (ADI) under accelerated storage conditions of 30° C. at 75% relative humidity for 6 months.

14. The varenicline formulation of claim 11, wherein the level of N-nitrosovarenicline (NNV) impurity remains about 2-fold below acceptable daily intake (ADI) under accelerated storage conditions of 40° C. at 75% relative humidity for 6 months.

15. The varenicline formulation of claim 10, wherein the formulation prevents further N-nitrosovarenicline (NNV) impurity formation after three months.

16. The varenicline formulation of claim 10, wherein the varenicline formulation contains approximately 0.5 mg varenicline tartrate per tablet.

17. The varenicline formulation of claim 10, wherein the varenicline formulation contains approximately 1.0 mg varenicline tartrate per tablet.

18. A varenicline formulation consisting of:
a tablet core consisting of:
(a) about 0.85% varenicline tartrate;
(b) about 60.50% microcrystalline cellulose;
(c) about 33.33% dibasic calcium phosphate anhydrous;
(d) about 2.00% croscarmellose sodium;
(e) about 2.57% propyl gallate;
(f) about 0.75% magnesium stearate,
wherein the amounts are weight percentages based on the total weight of the tablet core; and
a film coating.

19. The varenicline formulation of claim 18, wherein the formulation maintains a level of N-nitrosovarenicline (NNV) impurity over the shelf-life of the formulation of not more than 18.5 ppm (37 ng/day) of acceptable daily intake (ADI).

20. The varenicline formulation of claim 19, wherein the level of N-nitrosovarenicline (NNV) impurity remains about 2-fold below acceptable daily intake (ADI) under accelerated storage conditions of 25° C. at 60% relative humidity for 6 months.

21. The varenicline formulation of claim 19, wherein the level of N-nitrosovarenicline (NNV) impurity remains about 2-fold below acceptable daily intake (ADI) under accelerated storage conditions of 30° C. at 75% relative humidity for 6 months.

22. The varenicline formulation of claim 19, wherein the level of N-nitrosovarenicline (NNV) impurity remains about 2-fold below acceptable daily intake (ADI) under accelerated storage conditions of 40° C. at 75% relative humidity for 6 months.

23. The varenicline formulation of claim 18, wherein the formulation prevents further N-nitrosovarenicline (NNV) impurity formation after three months.

24. The varenicline formulation of claim 18, wherein the varenicline formulation contains approximately 0.5 mg varenicline tartrate per tablet.

25. The varenicline formulation of claim 18, wherein the varenicline formulation contains approximately 1.0 mg varenicline tartrate per tablet.

26. (canceled)
